(19) United States
(12) Patent Application Publication
(10) Pub. No.: US 2016/0345900 A1
(43) Pub. Date: Dec. 1, 2016

(54) Calorie measuring capsule

(71) Applicant: Simon Xiansheng Xe, San Jose, CA (US)

(72) Inventor: Simon Xiansheng Xe, San Jose, CA (US)

(21) Appl. No.: 14/726,750

(22) Filed: Jun. 1, 2015

(51) Int. Cl. A61B 5/00; A61B 5/07; A61B 5/145; H04B 1/034

(52) U.S. Cl. CPC A61B 5/4866; H04B 1/034; A61B 5/073; A61B 5/6861; A61B 5/14532; A61B 5/7278; A61B 2560/0475

(57) ABSTRACT

A capsule 1 is adapted to be ingestable by a human and comprises a density sensor, a sugar level sensor and a weight sensor for determining the calorie intake.

### TECHNICAL FIELD

The description relates generally to a capsule that is adapted to be ingested by a human for collecting calorie intake data.

### BACKGROUND

People starts to pay more attention to their health. There are many contributing reasons to a person's health. Eating habit is one, if not the most, important reason to one's health. There are many books and software teaching people what to eat, how to eat. There are also many systems tracking what people eat. However, everyone is different and their digestion system is different. The same food may lead to different result on different people. This calorie measuring capsule gathers data from within the user's digestion system. By tracking this data, combining with other user-specific data, such as exercise activity, heart rate, sleeping time etc, it allows a more personal analysis on what kind of food is good for that person and what kind of life style is to be suggested.

The capsule can send measured data in real time wirelessly to be received and calculated by a receiver. The capsule can also have a microprocessor and a data storage device so that the data is stored and can be retrieved later in a monitored environment.

### SUMMARY

In accordance with a first aspect of the present invention there is provided a capsule adapted to be ingestable by a human and comprising a calorie measuring device for determining the calorie intake of the human.

In a preferred embodiment of the invention, the calorie measuring device comprises a density sensor, a weight sensor and a sugar level sensor.

In a further preferred embodiment, the capsule comprises a wireless data transmitting device. This allows measured data be sent in realtime through wireless signal.

In a further preferred embodiment, the capsule comprises a microprocessor and data storage device to process measured data, calculate calorie intake and store related data in the data storage device. This allows any data collected by the capsule to be extracted when the capsule is ejected.

Other features and advantages of the invention will become apparent to those skilled in the art upon review of the following detailed description, claims and drawings.

### BRIEF DESCRIPTION OF THE DRAWINGS

FIG. 1 is an end view of the capsule.

FIG. 2 is a horizontal cross sectional view of the capsule.

FIG. 3 is a vertical cross sectional view of the capsule.

FIG. 4 is a circuit diagram of the capsule.

FIG. 5 is a flowchart of the steps that take place when the capsule samples data and sends data.

### DETAILED DESCRIPTION

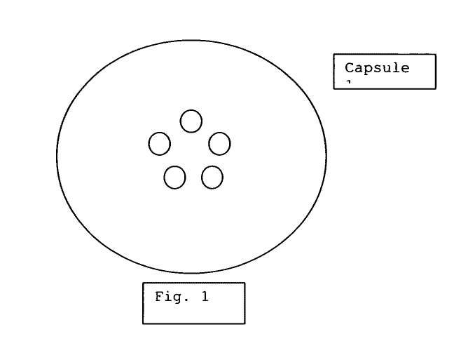

In FIG. 1, a capsule 1 has inlet and outlet holes on either end of the capsule. These holes allow fluid to flow into the capsule to be sampled by sensors. The capsule 1 is made of medical-grade polycarbonate, or any other suitable material, and is between approximately 16 mm and 41 mm long and between 6 mm and 16 mm in diameter.

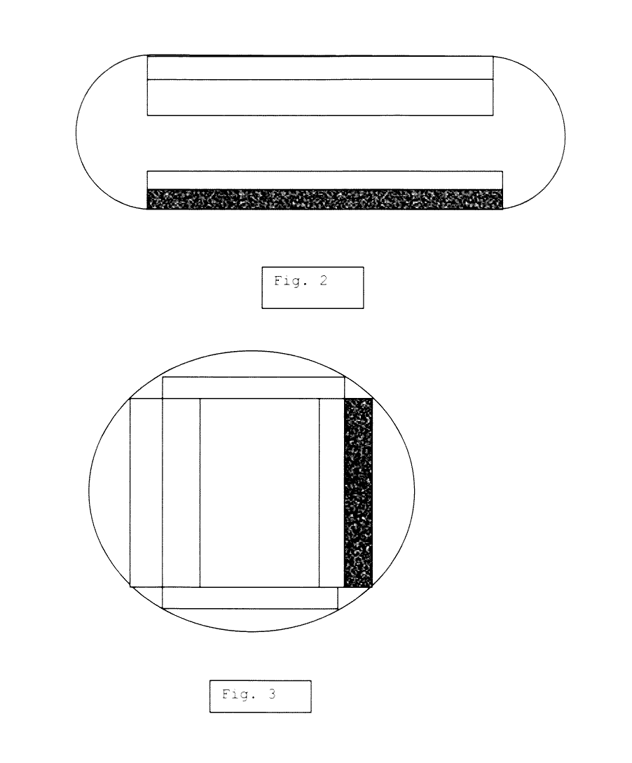

In FIG. 2, the horizontal section of the capsule is divided into five sections. Bottom section 1 is filled with high-density materials to ensure the capsule lays flat with the bottom section on the bottom. Density sensor section 2 houses density sensor. The density sensor measures the weight on the surface 2a G2a. Sample section 3 is for liquid to flow through. The volume of section 3 is fixed per capsule V3. The density of the liquid can be calculated as D=G2a/V3. Calorie sensor section 4 houses glucose sensor. The glucose sensor measures sugar level in the liquid through surface 4a. C4. Calorie index CI is calculated as: CI=C4×D×4. Weight sensor section 5 houses weight sensor.

The weight sensor measures weight on the outside surface of the capsule 1 G5. The calorie index CI and the weight G5 are both sent to receiver to be tracked.

In FIG. 3, the vertical section of the capsule is divided into seven sections. Sections 1-5 are described above. The transmitter section 6 houses a microprocessor 8 and a wireless transmitter 9. The microprocessor 8 converts sensor data into digital data and the transmitter 9 transmits data wirelessly to be received by a receiver. The microprocessor 8 is a AD7817 processor, although any suitable processor could be used as an alternative. The storage section 7 hoses a data storage device. The microprocessor sends converted sensor data to the storage device.

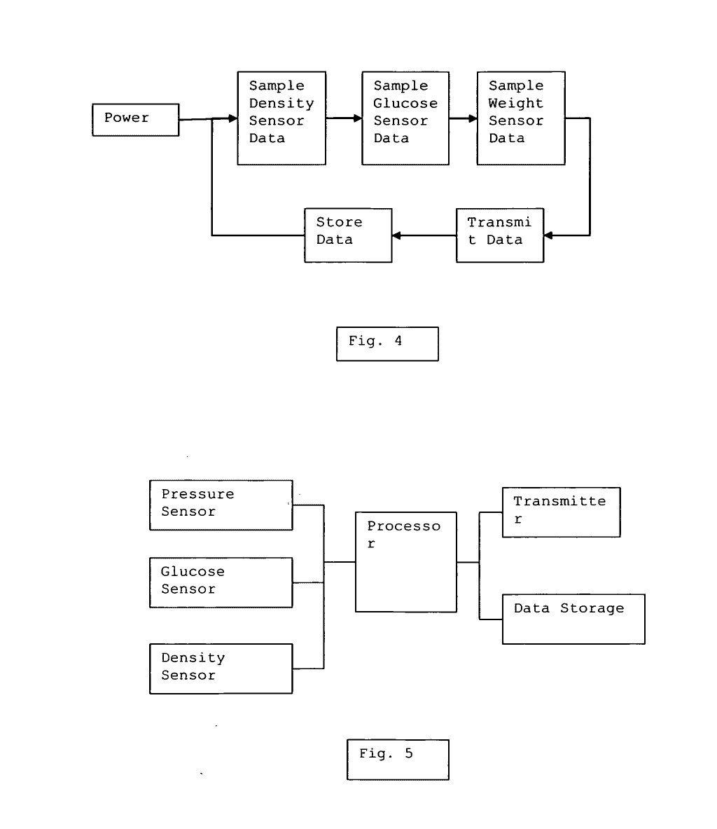

FIG. 4 shows a circuit diagram for the capsule. The density sensor, glucose sensor, weight sensor, wireless transmitter and data storage devices are all connected to the microprocessor 8.

FIG. 5 shows the steps that are followed when the capsule collects data and sends data.

JP2013204582

Calorie Measurement Device

PROBLEM TO BE SOLVED: To provide a calorie measurement device that prevents a value indicated in a calorie meter from slightly varying due to the influence of exhaust gas

JP2004267535

Calorie Consumption Measuring Meter

PROBLEM TO BE SOLVED: To provide a calorie consumption measuring meter estimating the quality of an exercise and precisely measuring the calorie consumption. SOLUTION: This calorie consumption . . .

EP1424038A1

Device for Measuring Calorie Expenditure

In order to obtain calorie expenditure with good accuracy, the device is provided with a basal metabolic state specifying element (142) which specifies the subject's basal metabolic state from his . . .

US20030014281

Calorie Management Apparatus

Disclosed is a calorie management apparatus for calculating and managing a calorie intake, comprising: an input unit; a storage unit; a display unit; and a control unit. According to the present . . .

JP51121386

Apparatus for Measuring a Calorie

PURPOSE: To provide an apparatus for measuring a calorie, which can cause heat of chemical reaction to easily and rapidly disperse, and continuous measuring to be

US20010017649

Capsule

System for producing a stereoscopic image of an object, and displaying the stereoscopic image, the system including a capsule and a control unit, the capsule including a sensor assembly, a . . .

1. A capsule adapted to be ingestable by a human and comprising a calorie measuring device for measuring calorie intake by that human.

2. A capsule as claimed in claim 1, wherein the calorie measuring device comprises a density sensor, a weight sensor and a sugar level sensor.

3. A capsule as claimed in claim 1, wherein the capsule additionally comprises a wireless data transmitter.

4. A capsule as claimed in claim 1, wherein the capsule additionally comprises a processor.

5. A capsule as claimed in claim 1, wherein the capsule additionally comprises a data storage device.

6. A method of calculating calorie intake from the measured density, weight and sugar level data.
